(19) United States
(12) Patent Application Publication
(10) Pub. No.: US 2026/0256504 A1
(43) Pub. Date: Sep. 3, 2026

(54) SCREW AND DRIVER SYSTEM AND ASSOCIATED METHODS

(71) Applicants: Axel Cremer, Fehrenkrug (DE); Richard Garret Mauldin, Erie, CO (US)

(72) Inventors: Axel Cremer, Fehrenkrug (DE); Richard Garret Mauldin, Erie, CO (US)

(21) Appl. No.: 19/489,453

(22) PCT Filed: May 31, 2024
(86) PCT No.: PCT/US2024/031953

§ 371 Date: Dec. 2, 2025

Related U.S. Application Data
(60) Provisional application No. 63/470,550, filed on Jun. 2, 2023.

(51) Int. Cl. A61B 17/86; A61B 17/16; A61B 17/88

(52) U.S. Cl. CPC A61B 17/864; A61B 17/1615; A61B 17/1655; A61B 17/8615; A61B 17/863; A61B 17/866; A61B 17/8888

(57) ABSTRACT

An orthopedic screw and driver system comprises an orthopedic screw having an elongate body extending along an axis from a screw distal end to a screw proximal end, the orthopedic screw having a canula extending from the screw distal end to the screw proximal end. A driver having a driver elongate body and a K-wire. The driver elongate body extends along an axis from a driver elongate body distal end to a. driver elongate body proximal end, the driver elongate body having a driver elongate body passageway extending from the elongate body distal end towards the elongate body proximal end. The K-wire extends along an axis from a distal end to a proximal end, and has a first portion and a second portion, the first portion having a tip, the second portion having an exterior surface complimentary to the canula, the second portion disposed in the driver elongate body passageway.

WO

### FIELD

The disclosure relates to the field of medical devices. More particularly, the disclosure relates to screw and driver systems useful for the insertion of orthopedic screws into bone.

### BACKGROUND

Orthopedic screws, commonly referred to as bone screws, are commonly employed to stabilize bone fragments, fixate implants, or facilitate bone healing in various medical procedures. An orthopedic screw driver is an essential tool used to install, remove, or facilitate bone healing in various medical procedures.

Conventional orthopedic screw systems typically consist of an orthopedic screw and a corresponding driver. The orthopedic screw typically features a threaded elongate body and a head that allows for engagement with the driver. In conventional systems, the driver generally comprises a handle, a shaft, and a screw engagement portion.

Although conventional orthopedic screw and driver systems have shown some success, they also possess certain limitations. For example, one common challenge is associated with driver engagement and disengagement. Once the orthopedic screw is placed at an insertion point, it can be difficult to align the screw engagement portion of the driver with the head of the orthopedic screw. The conventional orthopedic screw and driver systems often require multiple rotations or adjustments to secure or remove the driver from the screw head, which can be time-consuming and cumbersome during treatment procedures.

Another drawback to conventional orthopedic screw and driver systems is related to the torque transmission between the driver and the orthopedic screw. Conventional orthopedic screw and driver systems may encounter slippage or insufficient torque transfer during installation, leading to inadequate screw fixation, potential damage to surrounding tissues, or damage to the screw.

A need exists, therefore, for improved orthopedic screw and driver systems and associated methods.

### BRIEF SUMMARY OF SELECTED EXAMPLES

Various example screw and driver systems are described. Various example methods are also described.

An example screw and driver system comprises a screw having an elongate body extending axially along a longitudinal axis from a screw distal end to a screw proximal end, the screw having a non-circular canula extending from the screw distal end to the screw proximal end; and a driver having a driver elongate body and a K-wire; wherein the driver elongate body extends axially along a longitudinal axis from a driver elongate body distal end to a driver elongate body proximal end, the driver elongate body having a driver elongate body passageway extending from the elongate body distal end towards the elongate body proximal end; and wherein the K-wire extends axially along a longitudinal axis from a K-wire distal end to a K-wire proximal end, the K-wire having a K-wire first portion and a K-wire second portion, the K-wire first portion having a non-threaded tip with a cutting edge, the K-wire second portion having a non-circular shaped exterior surface that is complimentary to the non-circular canula of the screw, the K-wire second portion disposed in the driver elongate body passageway.

Another example screw and driver system comprises a screw having an elongate body extending axially along a longitudinal axis from a screw distal end to a screw proximal end, the screw having a non-circular canula extending from the screw distal end to the screw proximal end; and a driver having a driver elongate body, a sleeve, and a K-wire; wherein the driver elongate body extends axially along a longitudinal axis from a driver elongate body distal end to a driver elongate body proximal end, the driver elongate body having a driver elongate body passageway extending from the elongate body distal end towards the elongate body proximal end; wherein the sleeve extends axially along a longitudinal axis from a sleeve distal end to a sleeve proximal end, the sleeve having a sleeve passageway extending from the sleeve distal end to the sleeve proximal end, the sleeve having a sleeve non-circular exterior surface complimentary to the driver elongate body passageway and the non-circular canula of the screw, the sleeve being disposed in the driver elongate body passageway; and wherein the K-wire extends axially along a longitudinal axis from a K-wire distal end to a K-wire proximal end, the K-wire having a K-wire first portion and a K-wire second portion, the K-wire first portion having a non-threaded tip, the K-wire second portion being complimentary to the sleeve passageway, the K-wire disposed in the sleeve passageway.

An example method of using a driver to insert an orthopedic screw into a bone comprises inserting a K-wire at an insertion point; drilling a hole in a bone with the K-wire; drilling a hole in a bone with a cannulated drill over the k-wire (minor diameter of screw); placing a screw on the K-wire; placing an elongate body on a distal end of the K-wire; rotating the elongate body to insert the screw into the bone; removing the elongate body and the K-wire from the bone. Another example method of using a driver to insert an orthopedic screw into a bone comprises drilling a hole in a bone to create an insertion point; inserting a K-wire at the insertion point; drilling a hole in a bone with a cannulated drill over the k-wire; placing a sleeve on the K-wire; placing a screw on the sleeve; placing an elongate body on a distal end of the sleeve; rotating the elongate body to insert the screw into the bone; removing the elongate body, the sleeve, and the K-wire from the bone.

Additional understanding of the inventive screw and driver, and associated methods, can be obtained by reviewing the detailed description of selected examples, below, and the referenced drawings.

### DESCRIPTION OF FIGURES

FIG. 1 is a perspective view of an example screw and driver system.

FIG. 2 is another perspective view of the screw and driver system illustrated in FIG. 1.

FIG. 3 is a perspective view of an example screw.

FIG. 4 is another perspective view of the screw illustrated in FIG. 3.

FIG. 5 is a partial perspective view of the screw illustrated in FIG. 3 disposed on an example K-wire.

FIG. 6 is another perspective view of the screw illustrated in FIG. 3 disposed on an example K-wire.

FIG. 7 is a perspective view of another example screw.

FIG. 8 is a perspective view of the screw illustrated in FIG. 7.

FIG. 9 is a perspective view of an example K-wire.

FIG. 10 is an enlarged partial perspective view of the K-wire illustrated in FIG. 9 with an alternative tip.

FIG. 11 is an enlarged partial perspective view of the K-wire illustrated in FIG. 9.

FIG. 12 is a partial perspective view of the K-wire illustrated in FIG. 9 disposed in an example driver elongate body.

FIG. 13 is a perspective view of another example K-wire.

FIG. 14 is a partial perspective view of an example driver elongate body.

FIG. 15 is a perspective sectional view of an example driver elongate body.

FIG. 16 is a flowchart illustration of an example method of using a driver to insert an orthopedic screw into a bone.

FIG. 17 is an exploded view of another example screw and driver system.

FIG. 18 is a partial perspective sectional view of the screw and driver system illustrated in FIG. 17.

FIG. 19 is a flowchart illustration of another example method of using a driver to insert an orthopedic screw into a bone.

### DETAILED DESCRIPTION OF SELECTED EXAMPLES

The following detailed description and the appended drawings describe and illustrate various example screw and driver systems and example methods of using a driver to insert an orthopedic screw into a bone. The description and illustration of these selected examples are provided to enable one skilled in the art to make and use example screw and driver systems and to perform example methods of using a driver to insert an orthopedic screw into a bone. They are not intended to limit the scope of the invention, or its protection, in any manner.

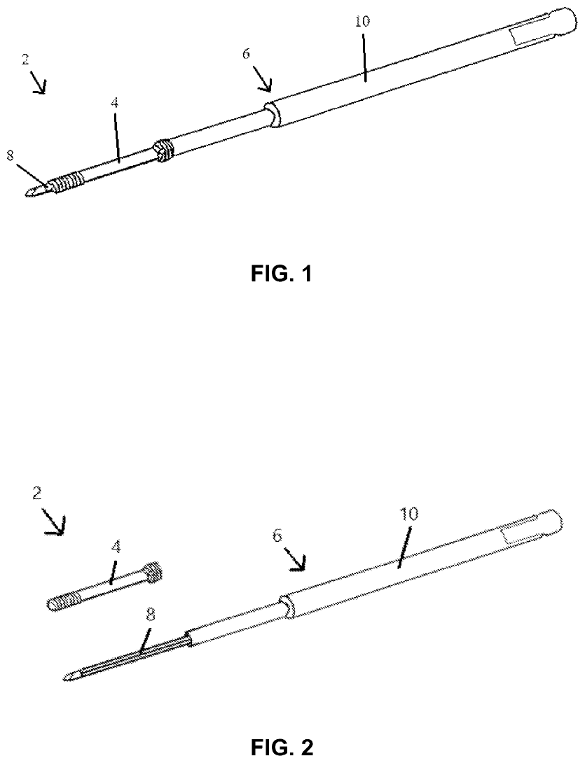

FIGS. 1 and 2 each illustrate a first example screw and driver system 2. The first screw and driver system 2 comprises a first example screw 4 and a first example driver 6. The first example driver comprises a first example K-wire 8 and a first example driver elongate body 10.

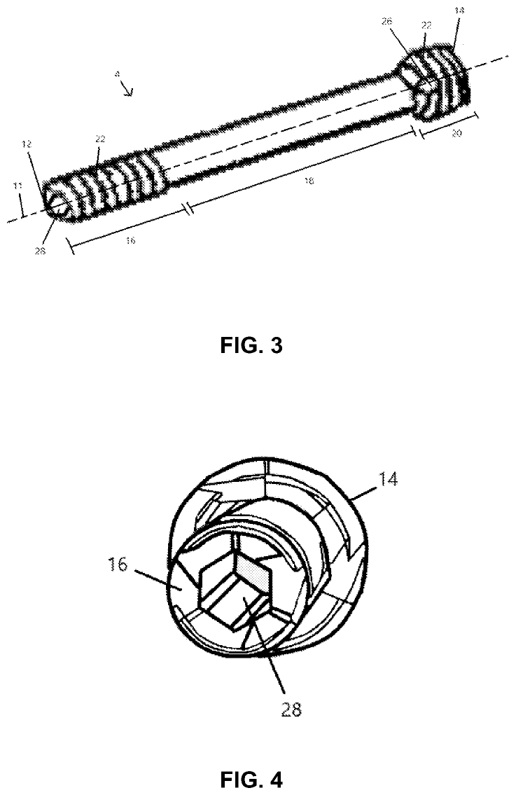

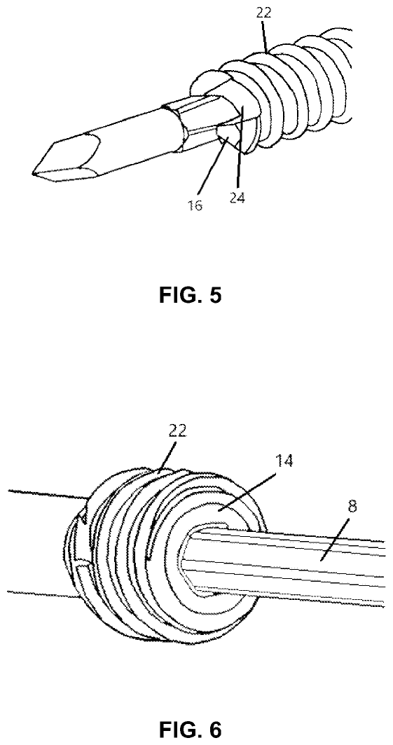

FIGS. 3, 4, 5, and 6 each illustrate the first example screw 4. The first example orthopedic screw 4 can be a bioabsorbable screw. The first example orthopedic screw 4 has a screw elongate body extending axially along a longitudinal axis 11 from a screw distal end 12 to a screw proximal end 14. The screw elongate body can have three portions including a screw distal portion 16, a screw intermediate portion 18, and a screw proximal portion 20. The screw distal portion 16 and the screw proximal portion 20 can have threads 22. The screw intermediate portion 18 can have a non-threaded exterior surface. Also, in other embodiments the screw intermediate portion 18 can have threads 22. The screw proximal portion 20 has a first circumference and the screw distal portion 16 has a second circumference. In some embodiments, the first circumference is larger than the second circumference; however, the first circumference does not have to be larger than the second circumference. The first circumference and second circumference can also have a relatively same sized circumference. In other embodiments, the first circumference can also be larger than the circumference of the screw intermediate portion 18. The screw distal portion 16 can also have a first screw cutout portion 24. The first screw cutout portion 24 can be seen best in FIG. 5. The screw proximal portion 20 can have a second screw cutout portion 26. The first example screw 4 has a canula 28 extending axially along the longitudinal axis 11 from the screw proximal end 20 to the screw distal end 16. The canula 28 is hex shaped in the first example screw 4; however, the canula 28 does not have to be hex shaped. The canula 28 can be any shape other than circular. Possible canula 28 shapes can include, but are not limited to, Torx-shaped, square-shaped, torq-shaped, hex-shaped, and trilobular shaped. As can be seen in FIG. 6, the first example screw 4 does not have a socket or recess because the first example screw and driver system 2 has no need for a socket or recess due to the canula 28 having a non-circular shape.

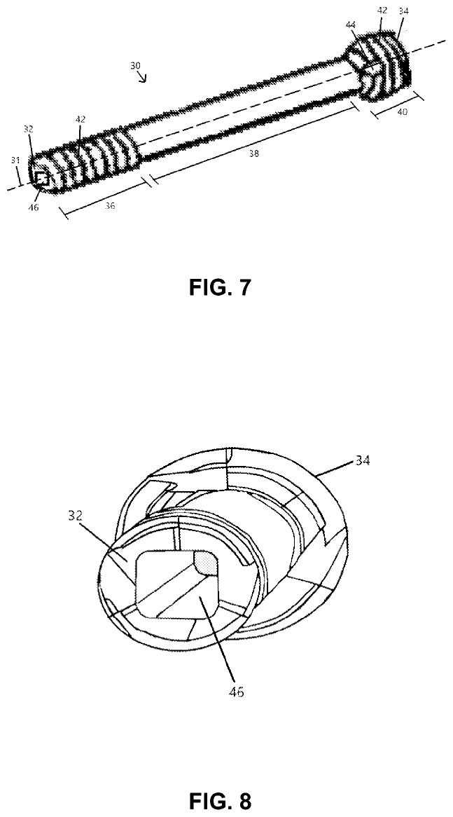

FIGS. 7 and 8 each illustrate a second example orthopedic screw 30. The second example orthopedic screw 4 can be a bioabsorbable screw. The second example screw 30 has a screw elongate body extending axially along a longitudinal axis 31 from a screw distal end 32 to a screw proximal end 34. The screw elongate body can have three portions including a screw distal portion 36, a screw intermediate portion 38, and a screw proximal portion 40. The screw distal portion 36 and the screw proximal portion 40 can have threads 42. The screw intermediate portion 38 can have a non-threaded exterior surface. Also, in other embodiments the screw intermediate portion 38 can have threads 22. The screw proximal portion 40 has a first circumference and the screw distal portion 36 has a second circumference. In some embodiments, the first circumference is larger than the second circumference; however, the first circumference does not have to be larger than the second circumference. The first circumference and second circumference can also have a relatively same sized circumference, In other embodiments, the first circumference can also be larger than the circumference of the screw intermediate portion 38. The screw proximal portion 40 can have a third screw cutout portion 44. The second example screw 30 has a canula 46 extending axially along the longitudinal axis 31 from the screw proximal end 34 to the screw distal end 32. The canula 46 is square shaped in the second example screw 30; however, the canula 46 does not have to be square shaped. It can be any shape other than circular. Possible canula 46 shapes can include, but are not limited to, Torx-shaped, square-shaped, torq-shaped, hex-shaped, and trilobular shaped. The second example screw 30 does not have a socket or recess because the second example screw 30 has no need for a socket or recess due to the canula 46 having a non-circular shape.

In all embodiments, the orthopedic screw can be made of any material suitable for use in medical devices intended for orthopedic use, including use as a long-term implant. Examples of suitable materials include metals, metal alloys, and polymeric materials. Examples of suitable metals include, but are not limited to, Titanium, Magnesium, and other metals. Examples of suitable metal alloys include, but are not limited to, Ti6Al4V, 316 LVM, 1.4441Ti-13Nb-13Zr, Ti-12Mo-6Zr-2Fe, Ti-15Mo-5Zr-3Al, Ti-15Mo, Ti-35Nb-7Zr-5Ta and Ti-29Nb-13Ta-4.6Zr Ti-6Al-7Nb and Ti-15Sn-4Nb-2Ta-0.2Pd Co—Cr—Mo alloys. Examples of suitable polymeric materials include, but are not limited to, polyaryletherketone (PAEK), polyether ether ketone (PEEK), PEEK (90G, 450G, 12, 14), Polyamide, PA66, carbon fiber reinforced polyaryletherketone (CFR PAEK), polyether ketone ketone (PEKK), carbon fiber reinforced polyether ketone ketone (CFR PEKK), carbon fiber reinforced polyether ether ketone (CFR PEEK), CFR PEEK (90G CA30, 90G CA20, 450G CA30, 450G CA20, I2 CF20, I2 CF30, I4 CF30, I4 CF20), Polyamide CFR, and PA66 CFR.

Orthopedic screws can include multiple components, such as an inner core member and an outer body member. In these embodiments, the components can be formed of the same or different materials. For example, an inner core member formed of a first metallic material, such as a metal or a metal alloy, and an outer body member formed of a second, different material, such as a polymeric material, a blended material such as a carbon fiber reinforced polymer, or another non-metallic material. It is considered particularly advantageous to include an inner core member formed of a Titanium alloy, such as Ti6Al4V, and an outer body member formed of a second, different material, such as CFR PEEK at least because this combination of materials provides desirable characteristics and a favorable balance between manufacturability and strength considerations, particularly considering the structural properties and engaging relationships with other components of the systems described herein. In these embodiments, the outer body member can be made of any material suitable for use in medical devices intended for orthopedic use, including use as a long-term implant. Examples of suitable types of materials include, but are not limited to, polymeric materials, blended materials such as carbon fiber reinforced polymers, and other materials. Examples of suitable polymeric materials include, but are not limited to, PAEK, CFR PAEK, PEKK, CFR PEKK, PEEK, CFR-PEEK, PEEK (90G, 450G, I2, I4), Polyamide, and PA66. Examples of suitable blended materials include, but are not limited to, PEEK-Carbon materials, CFR PAEK, CFR PEKK, CFR PEEK (90G CA30, 90G CA20, 450G CA30, 450G CA20, I2 CF20, I2 CF30, I4 CF30, I4 CF20), Polyamide CFR, PA66 CFR.

It is noted that the materials used in an orthopedic screw of a particular embodiment can include additives, coatings, fillers, and/or other elements if desired. For example, antibiotics, bioactive glass, silver, copper, or another material that can reduce bacterial colonization of the orthopedic screw following implantation can be included in the material of the inner core member, the outer body member, or both.

In some examples, the orthopedic screw is formed of a bioabsorbable material. Use of bioabsorbable materials in this manner is considered particularly advantageous at least because the structural arrangements of the screw, and the components of the systems, provide critical operational function for these relatively soft materials as compared to screws formed of metal. Indeed, the structural arrangements of the screws, and the components of the systems, can enable use of screws formed of bioabsorbable materials in anatomical locations and clinical situations in which use of conventional bioabsorbable screws may not be possible or desirable. For bioabsorbable screws according to the invention, any suitable bioabsorbable material can be used. Examples of suitable bioabsorbable materials include, but are not limited to, polyglycolic acid (PGA), polylactic acid (PLA), copolymers, such as mixtures of D- and L-isomers of PLA, combinations of PLA and PGA, and other copolymers, and some bioabsorbable metals including magnesium.

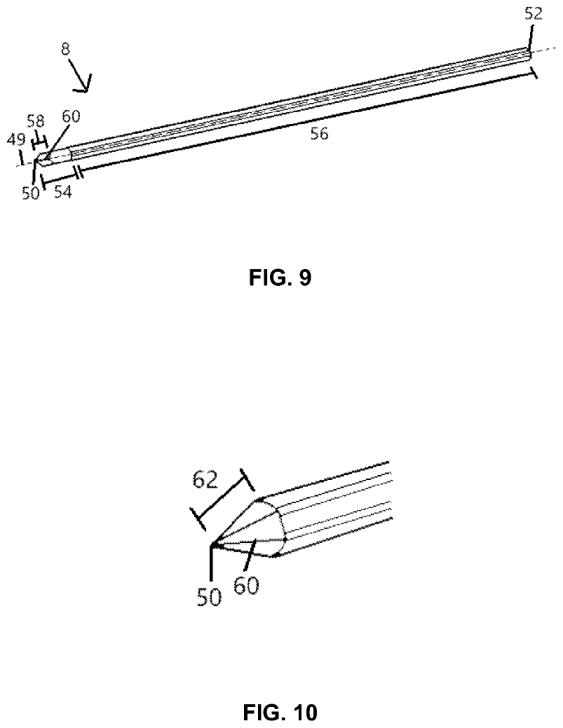

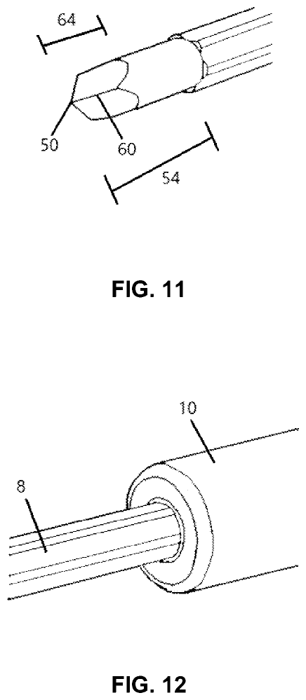

FIGS. 9, 10, 11, and 12 illustrate a first example K-wire 8 and a corresponding first example driver elongate body 48 of the first example screw and driver system 2. The first example K-wire 8 is part of the first example driver 6 of the screw and driver system 2. The first example K-wire 8 extends axially along a longitudinal axis 49 from a K-wire distal end 50 to a K-wire proximal end 52. The first example K-wire 8 has a K-wire first portion 54 and a K-wire second portion 56. The K-wire first portion 54 has a non-threaded tip 58 with a cutting edge 60. The non-threaded cutting tip 58 can be a variety of shapes including a hex tip 62 with six cutting edges 60, as seen in FIG. 10, a traditional tip 64, as seen in FIG. 11, for spinning when a screw advances, or any other desired tip with a cutting edge. The K-wire second portion 56 has a non-circular shaped exterior surface that is complimentary to the non-circular canula 28 of the first example screw 4. In this example, the K-wire second portion 56 is hex shaped; however, the K-wire second portion 56 can be any non-circular shape including, but not limited to, Torx-shaped, square-shaped, torq-shaped, hex-shaped, and trilobular shaped. At least a portion of the K-wire second portion 56 can be disposed in the first example driver elongate body 48 as can be seen in FIG. 12.

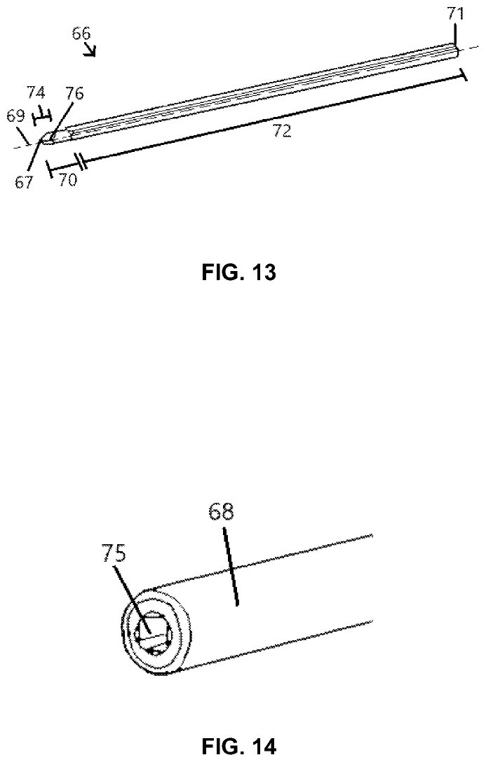

FIGS. 13 and 14 illustrate a second example K-wire 66 and a corresponding second example driver elongate body 68 of the first example screw and driver system 2. The second example K-wire 66 is part of the first example driver of the first example screw and driver system 2. The second example K-wire 66 extends axially along a longitudinal axis 69 from a K-wire distal end to a K-wire proximal end. The second example K-wire 66 has a K-wire first portion 70 and a K-wire second portion 72. The K-wire first portion 70 has a non-threaded tip 74 with a cutting edge 76. The non-threaded cutting tip 74 can be a variety of shapes including, but not limited to, a hex tip 62 with six cutting edges and a traditional tip for spinning when the screw advances, or any other desired tip with a cutting edge. The K-wire second portion 72 gas a non-circular shaped exterior surface that is complimentary to the non-circular canula 46 of the second example screw 30. The K-wire second portion 72 in this example is square shaped. The K-wire second portion 72 can have any non-circular shape including, but not limited to: Tox shaped, Hex shaped, Torq shaped, and Tri-lobular shaped. At least a portion of the K-wire second portion 72 can be disposed in a driver elongate body passageway 75 of the second example driver elongate body 68.

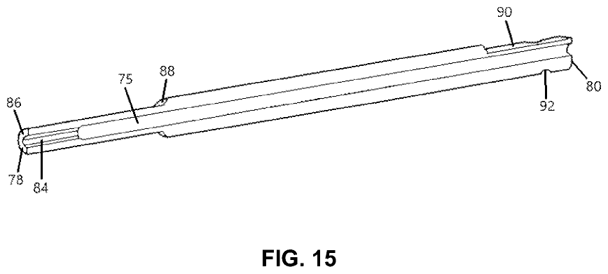

FIG. 15 illustrates a cross sectional view of the first example driver elongate body 10. The first example driver elongate body 10 extends axially along a longitudinal axis 77 from a driver elongate body distal end 78 to a driver elongate body proximal end 80. The first example driver elongate body 10 has a driver elongate body passageway 82 extending from the driver elongate body distal end 78 towards the driver elongate proximal end 80. At least a portion of the elongate body passageway 82 is faceted and is complimentary to the non-circular external surface of the first example K-wire 8. In the first example elongate body 10 illustrated there is a faceted portion 84 of the driver elongate body passageway 82. The faceted portion 84 is hex shaped. Although FIG. 15 illustrates that the faceted portion 48 as hex shaped, any non-circular shape will work, so long as it is the same shape and size as the K-wire proximal end 52. Also, although the illustrated example depicts only a portion of the elongate body passageway being faceted, the entire driver elongate body passageway 82 could be faceted. The first example elongate body 10 has a first shoulder 86 on the driver elongate body distal end 78 that can come in contact with the first example screw 4 when using the first example screw and driver system 2. The first example driver elongate body 10 can also have a second shoulder 88 that is proximal to the first shoulder 86. The first example driver elongate body 10 can also have a first section 90 and a second section 92 for the securement to a drill or other type of power tool

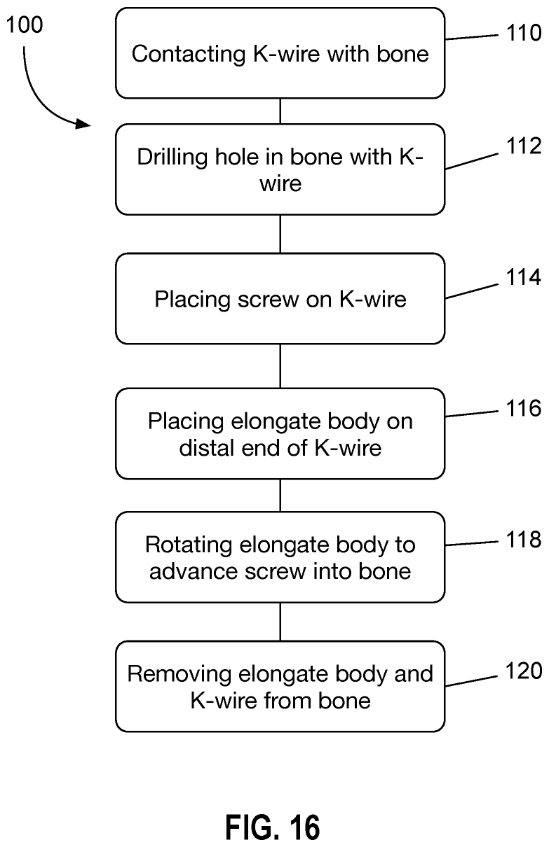

FIG. 16 illustrates a flowchart of a method of using the first example screw and driver system 2. If desired, a hole can be pre-drilled with a drill. However, it is not necessary to pre-drill a hole due to the design of the K-wire. Assuming no hole is pre-drilled, the K-wire is inserted at an insertion point on a bone. Next, a hole can be drilled in the bone using the K-wire. After the whole is drilled, a screw can be placed on the K-wire. After the placement of the screw onto the K-wire, the elongate body is disposed on the distal end of the K-wire. The elongate body is then rotated to insert the screw into the bone. Once the screw has been installed into the bone, the elongate body and the K-wire are removed from the bone. Optionally, the screw and the elongate body can be placed on the K-wire prior to drilling the hole in the bone. This method is advantageous at least because the method allows a K-wire to be inserted at an insertion point prior to placing the screw on the driver which would eliminate any stress or damage that can be caused to tissues or the bone from trying to get the screw with threads into a desired position. Further, this method allows for only one instrument to be used to install an orthopedic screw into a bone. With only one instrument being used, there is a decreased chance of causing unnecessary injury to surrounding tissues or the bone. This system and method are also advantageous at least because torque is distributed through the length of the screw rather than just a recessed socket. This distribution of torque can prevent damage to the screw, which can be particularly advantageous with non-metal screws and screws comprising relatively soft materials, such as polymeric materials, bioabsorbale materials, and other non-metal materials.

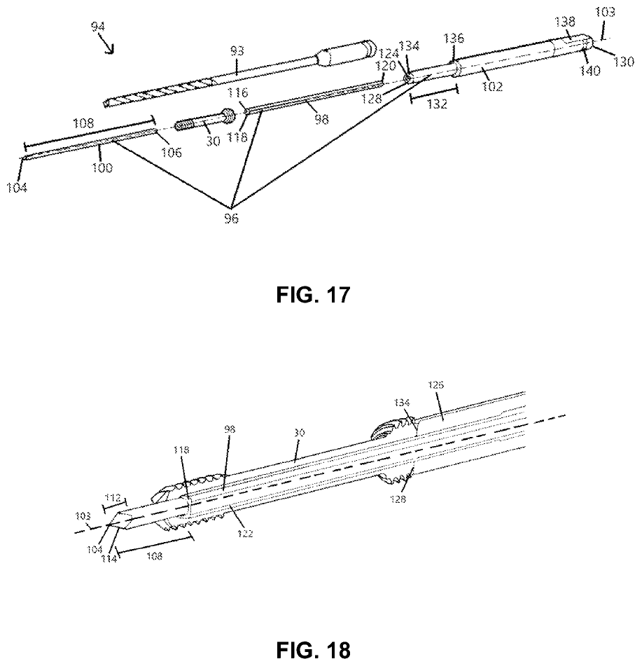

FIGS. 17 and 18 each illustrate a second example screw and driver system 94. The second example screw and driver system 94 comprises the second example screw 30 and a second example driver 96.

The screw can be any type of orthopedic screw, but the first example screw 4 and the second example screw 30 are the preferred screws. The only difference between the screws in the first screw and driver system 2 and the second screw and driver system 94 is that the screw in the second example screw and driver system 94 is shaped to compliment a sleeve 98 rather than the second example K-wire 66.

The second example driver 96 includes a third example K-wire 100, the sleeve 98, and a second example driver elongate body 102. The third example K-wire 100 extends axially along a longitudinal axis 103 from a K-wire distal end 104 to a K-wire proximal end 106. The third example K-wire 100 can have a K-wire proximal portion 108 and a K-wire distal portion 110. The K-wire distal portion 110 can have a non-threaded tip 112 with a cutting edge 114. The non-threaded cutting tip 112 can be a variety of shapes including a hex tip with six cutting edges, a traditional tip for spinning when a screw advances, or any type of tip that is desired. In this example, the K-wire proximal portion 108 is circular shaped. However, the K-wire proximal portion 108 can have any shape including, but not limited to, Torx-shaped, square-shaped, torq-shaped, hex-shaped, and trilobular shaped, as explained above regarding the first example screw and driver system 2. At least a portion of the third example K-wire 100 can be disposed in a sleeve passageway 116 as can be seen in FIG. 18.

The sleeve 98 of the second example driver 96 extends axially along a longitudinal axis 117 from a sleeve distal end 118 to a sleeve proximal end 120. Inside of the sleeve 98 is the sleeve passageway 116 extending from the sleeve distal end 118 to the sleeve proximal end 120. Although FIG. 17 depicts the sleeve passageway 116 as being circular shaped, it can be any shape including, but not limited to, Torx-shaped, square-shaped, torq-shaped, hex-shaped, and trilobular shaped. The sleeve 98 has a sleeve non-circular exterior surface 122 that is complimentary to a third example driver elongate body non-circular passageway 124. Although the sleeve 98 illustrated is depicted as having a square shaped exterior surface, the exterior surface can be any shape including, but not limited to, Torx-shaped, square-shaped, torq-shaped, hex-shaped, and trilobular shaped.

A third example driver elongate body 126 extends axially along a longitudinal axis 127 from a driver elongate body distal end 128 to a driver elongate body proximal end 130. The third example driver elongate body 126 has the driver elongate body passageway 124 extending from the driver elongate body distal end 128 towards the driver elongate body proximal end 130. The driver elongate body non-circular passageway 124 has a faceted portion 132. In the illustrated example, the driver elongate body non-circular passageway 124 has a square shaped faceted portion 132. Although the faceted portion 132 is depicted as square shaped in this example, any non-circular shape is acceptable, so long as the faceted portion 132 complimentary to the sleeve non-circular external surface 122. The third example driver elongate body 126 has a first shoulder 134 that can come in contact with the second example screw 30 when using the second screw and driver system 94. The third example driver elongate body 126 can also have a second shoulder 169 that is proximal to a first shoulder 134. The third example driver elongate body 126 can also have a first section 138 and a second section 140 for the securement to a drill 93 or other type of power tool.

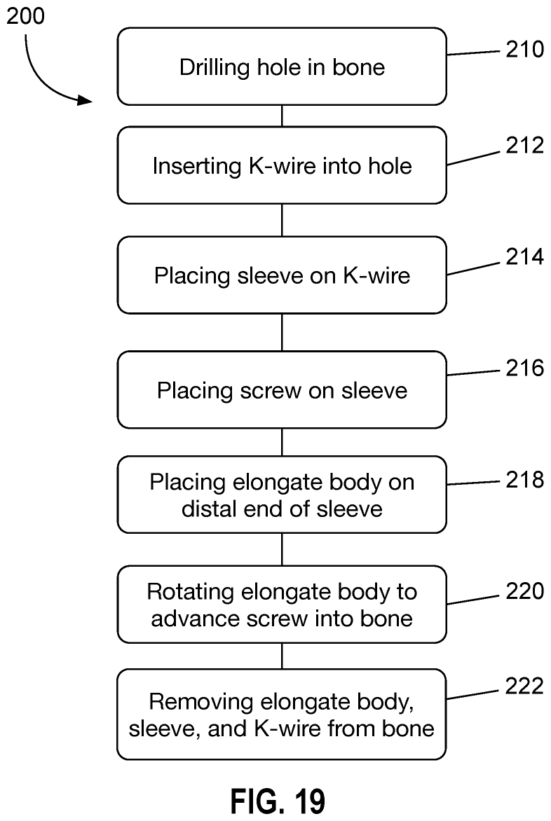

FIG. 19 illustrates a method of using the second example screw and driver system 94. If desired, a hole can be pre-drilled with the drill 93. However, it is not always necessary to pre-drill a hole due to the previous mentioned designs of the K-wire. Assuming a hole is pre-drilled, the K-wire is inserted at an insertion point, defined by the pre-drilled hole, on a bone. Next, the sleeve is placed on the K-wire. After the sleeve has been placed on the K-wire, the screw is placed on the sleeve. The driver elongate body is placed on the distal end of the sleeve. The driver elongate body is then rotated to insert the screw into the bone. Once the screw has been inserted into the bone, the elongate body, the sleeve, and the K-wire are removed from the bone. Optionally, no pre-drilling is necessary and a method similar to the method in FIG. 16 may be utilized. This method is advantageous at least because the method allows a K-wire to be inserted at an insertion point prior to placing the screw on the driver which would eliminate any stress or damage that can be caused to tissues or the bone from trying to get the screw with threads into a desired position. This system and method are also advantageous at least because torque is distributed through the length of the screw rather than just a recessed socket. This distribution of torque prevents damage to the screw caused by bioabsorbable screws being made of a softer material than orthopedic screws made out of hard metals.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

We claim:

1. An orthopedic screw and driver system, comprising:
an orthopedic screw having an elongate body extending axially along a longitudinal axis from a screw distal end to a screw proximal end, the orthopedic screw having a non-circular canula extending from the screw distal end to the screw proximal end; and
a driver having a driver elongate body and a K-wire;
wherein the driver elongate body extends axially along a longitudinal axis from a driver elongate body distal end to a driver elongate body proximal end, the driver elongate body having a driver elongate body passageway extending from the elongate body distal end towards the elongate body proximal end; and
wherein the K-wire extends axially along a longitudinal axis from a K-wire distal end to a K-wire proximal end, the K-wire having a K-wire first portion and a K-wire second portion, the K-wire first portion having a non-threaded tip with a cutting edge, the K-wire second portion having a non-circular shaped exterior surface that is complimentary to the non-circular canula of the screw, the K-wire second portion disposed in the driver elongate body passageway.

2. The orthopedic screw and driver system of claim 1, wherein the orthopedic screw comprises a bioabsorbable material.

3. The orthopedic screw and driver system of claim 1, wherein the orthopedic screw comprises a polymeric material.

4. The orthopedic screw and driver system of claim 3, wherein the polymeric material comprises PEEK.

5. The orthopedic screw and driver system of claim 1, wherein the canula of the orthopedic screw is hex-shaped.

6. The orthopedic screw and driver system of claim 1, wherein the canula of the orthopedic screw is square-shaped.

7. The orthopedic screw and driver system of claim 1, wherein the canula of the orthopedic screw is Torx-shaped, square-shaped, torq-shaped, hex-shaped, or trilobular shaped.

8. The orthopedic screw and driver system of claim 1, wherein the orthopedic screw has a screw distal portion, a screw intermediate portion, and a screw proximal portion;
wherein the screw intermediate portion has a non-threaded exterior surface.

9. The orthopedic screw and driver system of claim 4, wherein the screw distal portion has a first circumference and the screw proximal portion has a second circumference;
wherein the second circumference is larger than the first circumference.

10. The orthopedic screw and driver system of claim 1, wherein the orthopedic screw has a screw distal portion, a screw intermediate portion, and a screw proximal portion;
wherein the screw distal portion has a first circumference and the screw proximate portion has a second circumference, wherein the second circumference is larger than the first circumference.

11. The orthopedic screw and driver system of claim 1, wherein the orthopedic screw has a first screw cutout portion.

12. The orthopedic screw and driver system of claim 11, wherein the screw has a second screw cutout portion.

13. The screw and driver system of claim 12, wherein the screw proximal portion has a first circumference that is larger than the circumference of the screw intermediate portion.

14. The orthopedic screw and driver system of claim 1, wherein the driver elongate body passageway has a faceted portion and a non-faceted portion.

15. The screw and driver system of claim 1, wherein the driver elongate body has a first section and a second section configured to secure the driver elongate body to a drill.

16. The screw and driver system of claim 1, wherein the driver elongate body has a first shoulder.

17. The screw and driver system of claim 14, wherein the driver elongate body has a second shoulder.

18. The screw and driver system of claim 1, wherein the K-wire first portion has a hex tip.

19. An orthopedic screw and driver system, comprising:
an orthopedic screw having an elongate body extending axially along a longitudinal axis from a screw distal end to a screw proximal end, the screw having a non-circular canula extending from the screw distal end to the screw proximal end; and
a driver having a driver elongate body, a sleeve, and a K-wire;
wherein the driver elongate body extends axially along a longitudinal axis from a driver elongate body distal end to a driver elongate body proximal end, the driver elongate body having a driver elongate body passageway extending from the elongate body distal end towards the elongate body proximal end;
wherein the sleeve extends axially along a longitudinal axis from a sleeve distal end to a sleeve proximal end, the sleeve having a sleeve passageway extending from the sleeve distal end to the sleeve proximal end, the sleeve having a sleeve non-circular exterior surface complimentary to the driver elongate body passageway and the non-circular canula of the orthopedic screw, the sleeve being disposed in the driver elongate body passageway, and
wherein the K-wire extends axially along a longitudinal axis from a K-wire distal end to a K-wire proximal end, the K-wire having a K-wire first portion and a K-wire second portion, the K-wire first portion having a non-threaded tip, the K-wire second portion being complimentary to the sleeve passageway, the K-wire disposed in the sleeve passageway.

20. A method of using a driver to insert an orthopedic screw into a bone comprising:
inserting a distal end of a K-wire at an insertion point in a bone, the K-wire defining a non-circular circumferential surface;
drilling a hole in the bone with the K-wire;
passing an orthopedic screw over a proximal end of the K-wire, the orthopedic screw defining a lumen and having an interior, non-circular circumferential surface that is complimentary and mating with the non-circular circumferential surface of the K-wire;
placing an elongate body on the proximal end of the K-wire, the elongate body defining a lumen and having an interior, non-circular circumferential surface that is complimentary and mating with the non-circular circumferential surface of the K-wire;
rotating the elongate body to rotate the K-wire and the orthopedic screw and drive the orthopedic screw into the bone;
while rotating the elongate body, pushing the elongate body distally while the distal end of the elongate body contacts a proximal end of the orthopedic screw;
removing the elongate body and the K-wire from the bone, leaving the orthopedic screw in the bone.
